(19) United States
(12) Patent Application Publication
(10) Pub. No.: US 2022/0062577 A1
(43) Pub. Date: Mar. 3, 2022

(54) VENTILATOR ASSEMBLY AND MIXING SYSTEM THEREFOR

(71) Applicant: Kelly Pneumatics, Inc., Newport Beach, CA (US)

(72) Inventor: Edmund F. Kelly, Costa Mesa, CA (US)

(21) Appl. No.: 17/459,016

(22) Filed: Aug. 27, 2021

Related U.S. Application Data
(60) Provisional application No. 63/071,239, filed on Aug. 27, 2020.

(51) Int. Cl. A61M 16/12; A61M 16/20; A61M 16/00

(52) U.S. Cl. CPC A61M 16/12; A61M 2205/3334; A61M 16/009; A61M 16/205

(57) ABSTRACT

A gas mixing system that includes first and second flow assemblies and an exhaust gas outlet. Each of the first and second flow assemblies include a plurality of on/off valves with different flow rate values and a proportional valve. The first flow assembly is configured to have a first gas flowed therethrough and the second flow assembly is configured to have a second gas flowed therethrough. The output of the first flow assembly is combined with the output of the second flow assembly and the mixed gases exit through the exhaust gas outlet. The gas mixing system can be used in a ventilator assembly.

### CROSS-REFERENCE TO RELATED APPLICATIONS

This application claims the benefit of U.S. Provisional Application No. 63/071,239, filed on Aug. 27, 2020, which is incorporated by reference herein in its entirety.

### FIELD OF THE INVENTION

The present invention relates to a gas mixing system, and particularly, a gas mixing system that can be used with a ventilator.

### BACKGROUND OF THE INVENTION

Considering the current world pandemic, a need exists for a low cost, reliable rescue ventilator that does not require a conventional blender or use a flow sensor as most ventilators in the world use one or the other or both. A ventilator that does not rely on an overburdened supply chain is desirable.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with a first aspect of the present invention, there is provided a gas mixing system that includes first and second flow assemblies and an exhaust gas outlet. The first flow assembly includes an inlet for receiving a first gas, a regulator, a plurality of on/off valves connected in series and a proportional valve. Each on/off valve includes a different on/off flow rate value and is selectively openable to allow the first gas to flow therethrough. A first gas on/off flow path is defined from the inlet, through the regulator, through at least one of the on/off valves and to a first gas on/off outlet. A first gas proportional flow path is defined from the inlet, through the regulator, through the proportional valve and to a first gas proportional outlet. The first gas on/off flow path and first gas proportional flow path combine to create a first gas output path. The second flow assembly includes an inlet for receiving a second gas, a regulator, a plurality of on/off valves connected in series and a proportional valve. Each on/off valve includes a different on/off flow rate value and is selectively openable to allow the second gas to flow therethrough. A second gas on/off flow path is defined from the inlet, through the regulator, through at least one of the on/off valves and to a second gas on/off outlet. A second gas proportional flow path is defined from the inlet, through the regulator, through the proportional valve and to a second gas proportional outlet. The second gas on/off flow path and second gas proportional flow path combine to create a second gas output path. mixed gas outlet, wherein the first gas output path and second gas output path combine to create an exhaust gas outlet path that extends through the exhaust gas outlet.

The present invention also includes a method of mixing a first gas or fluid and a second gas or fluid that includes flowing a first gas through an inlet of a first flow assembly and flowing a second gas through an inlet of a second flow assembly.

The first flow assembly includes the inlet, at least first and second on/off valves and a proportional valve, wherein the first on/off valve has a first on/off flow rate value and the second on/off valve has a second on/off flow rate value, wherein the proportional valve has a range of proportional flow rate values. Flowing the first gas along a first gas on/off flow path, which extends through at least one of the first or second on/off valves and through an on/off outlet at a first gas on/off flow rate. Flowing the first gas along a first gas proportional path, which extends through the proportional valve and through a proportional outlet at a first gas proportional flow rate. The on/off outlet and proportional outlet are in flow communication such that the first gas on/off flow path meets the first gas proportional flow path to form a first gas output path where the first gas flows at a first gas combined flow rate.

The second flow assembly includes the inlet, at least first and second on/off valves and a proportional valve, wherein the first on/off valve has a first on/off flow rate value and the second on/off valve has a second on/off flow rate value, wherein the proportional valve has a range of proportional flow rate values. Flowing the second gas along a second gas on/off flow path, which extends through at least one of the first or second on/off valves and through an on/off outlet at a second gas on/off flow rate. Flowing the second gas along a second gas proportional path, which extends through the proportional valve and through a proportional outlet at a second gas proportional flow rate. The on/off outlet and proportional outlet are in flow communication such that the second gas on/off flow path meets the second gas proportional flow path to form a second gas output path where the second gas flows at a second gas combined flow rate.

The first gas output path is in flow communication with the second gas output path to form a mixed gas output path where the first gas at the first gas combined flow rate mixes with the second gas at the second gas combined flow rate to form an exhaust gas that flows to and through the mixed gas outlet or outlet system port at the predetermined exhaust gas flow rate. In a method where the system is mixing air and oxygen, the exhaust gas includes a predetermined FiO₂ or oxygen concentration and a predetermined exhaust gas flow rate. In use, the system software calculates the second gas (oxygen) flow rate (also referred to as the oxygen flow assembly output flow rate) via the equation (predetermined exhaust gas flow rate) multiplied by (predetermined FiO₂−21) divided by 79. The controller then communicates the proper commands to open a combination of one or more on/off valves and possibly the proportional valve to achieve the oxygen flow rate. Then the system software calculates the first gas (air) flow rate (also referred to as the air flow assembly output flow rate) via the equation predetermined exhaust flow rate—oxygen flow assembly output flow rate. The controller then communicates the proper commands to open a combination of one or more on/off valves and possibly the proportional valve to achieve the air flow rate. The air flow rate and oxygen flow rate are combined to achieve the predetermined exhaust gas flow rate at the predetermined FiO₂.

The present invention includes a valve system for the hybrid flow and blending of at least two gases that can be used with a ventilator or in other situations or scenarios where blended gases are desired. In an exemplary embodiment (and as is used in a ventilator scenario), the system described herein uses oxygen and air as the gases to be blended. However, the same method and system can be used on any other combination of gases. Also, the described system and method can be used to blend or mix more than two gases.

The individual hybrid flow for each gas is accomplished by the use of binary flow utilizing on/off solenoid valves or other on/off valves, in conjunction with proportional flow from one proportional solenoid valve.

In a preferred embodiment, the system includes, but is not limited to, twelve on/off solenoid valves and two proportional solenoid valves. There are six on/off valves and one proportional valve used for each gas. The proportional valve system allows fine tuning of the gases as they are mixed. All from the same source and all go to the same downstream port to create one flow rate.

In an exemplary embodiment, the valve system is used with a ventilator, where the mixed gases are delivered via a ventilator to a patient. The present invention includes a ventilator assembly and a mixing system that provides binary, hybrid or proportional flow that delivers an accurate desired flow rate of a gas at relatively low flow rates and with relatively low error rates such that it preferably eliminates the need for the use of a flow sensor. Further, a mixed gas of desired concentration can be relatively accurately delivered by running and coordinating two “hybrid flow” mechanisms (referred to herein as first and second flow assemblies) at various flow rates and then combining their output flows to produce the mixed gas at the desired concentration, eliminating the need in a preferred embodiment for both a flow sensor and a mixer/blender. The mixed gas can then be delivered to the patient utilizing the ventilator. In another embodiment, a flow sensor and a mixer/blender can be used.

### BRIEF DESCRIPTION OF THE DRAWINGS

The invention may be more readily understood by referring to the accompanying drawings in which:

FIG. 1 is perspective view of a ventilator assembly in accordance with a preferred embodiment of the present invention;

FIG. 2 is a flow diagram of the mixing system;

FIG. 3 is a schematic of the mixing system;

FIG. 4 is an exploded perspective view of the ventilator assembly;

FIG. 5 is a perspective view of the mixing system; and

FIG. 6 is an electric schematic of a ventilator system that includes the ventilator assembly and mixing system.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

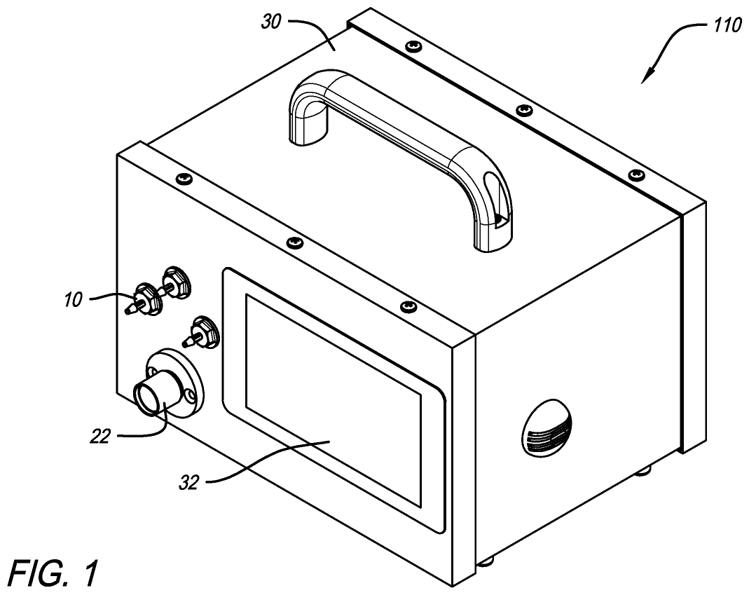

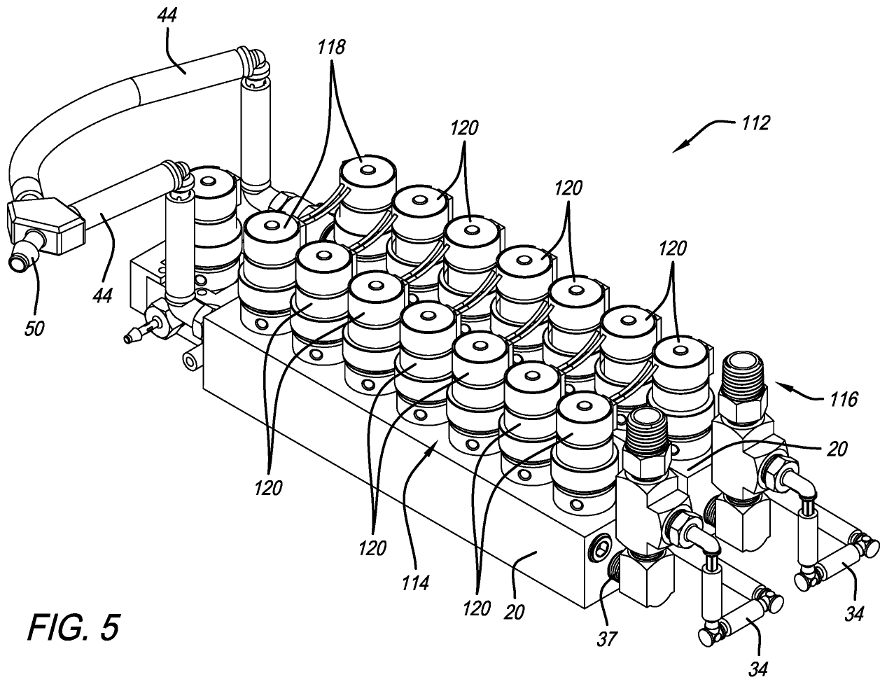

Referring now to the drawings, wherein the showings are for purposes of illustrating the present invention and not for purposes of limiting the same, FIGS. 1-6 show embodiments of a ventilator assembly 110 and a mixing system or mixing assembly 112 that provide binary, hybrid or proportional flow that delivers an accurate desired flow rate of a gas at relatively low flow rates and with relatively low error rates such that it preferably eliminates the need for the use of a flow sensor. Further, a mixed gas of desired concentration can be relatively accurately delivered by running and coordinating two “hybrid flow” mechanisms (referred to herein as first and second flow assemblies 114 and 116) at various flow rates and then combining their output flows to produce the mixed gas at the desired concentration, preferably eliminating or minimizing the need for both a flow sensor and a mixer/blender. In an embodiment where the mixing assembly 112 is used in a ventilator, such as ventilator assembly 110, the mixed gas can then be delivered to the patient utilizing the ventilator.

In a preferred embodiment, the mixed gas output is achieved by mixing the output of the first and second flow assemblies 114 and 116. Each of the first and second flow assemblies 114 and 116 preferably include at least one proportional control flow valve 118 and multiple or a plurality of on-off valves 120 that can be activated selectively, additively to, and in parallel with each other and the proportional valve 118 in a manifold block 20 to achieve a variety of flow rates.

In an exemplary embodiment, the flow rate from the proportional control flow valve 118 is usually in the lower range of flow rates (0-2 liters per minute, lpm or l/min) and can be varied within its parameters (0.01 lpm or even less). In use, the on-off valves 120 each include a different flow rate value (e.g., they allow a different lpm therethrough) and are turned on or off (include an on state and an off state) to add or subtract from the overall flow rate flowing through the manifold. The first and second flow assemblies 114 and 116 are supplied with different gas sources (e.g., pure oxygen and air) and are simultaneously controlled as to their respective flow rates so that the combination of their respective output gases achieve the desired concentration of the combined gas across a range of flow rates.

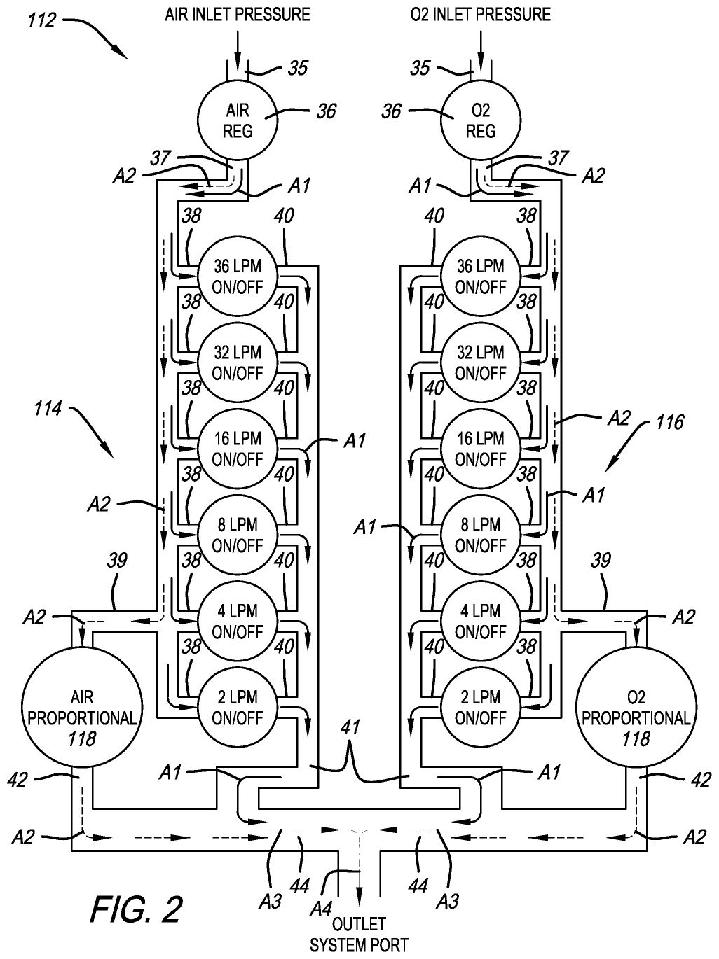

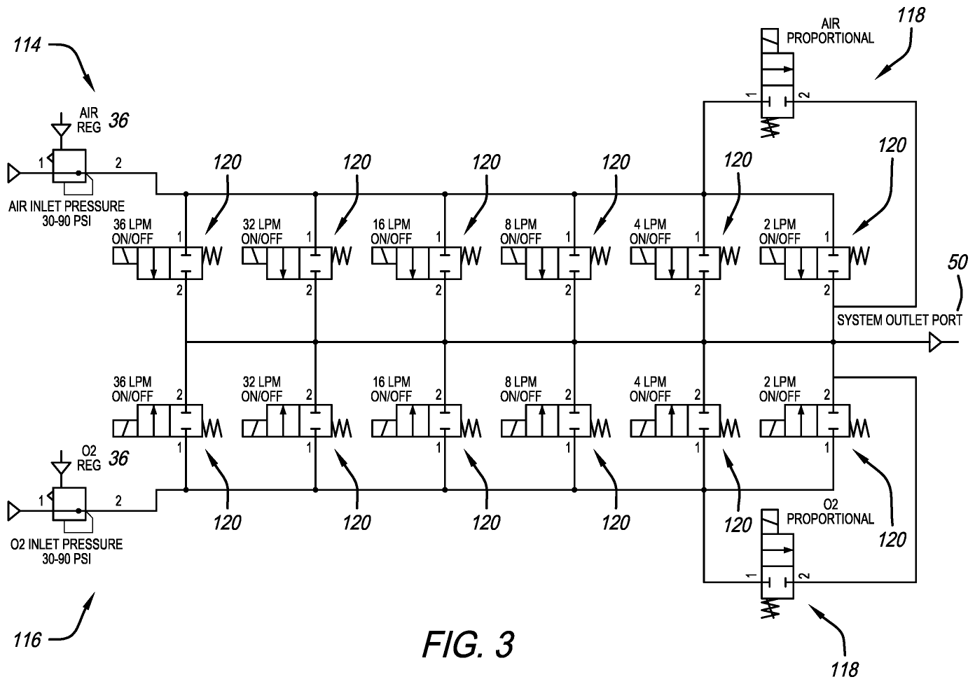

In the exemplary embodiment shown in the drawings (e.g., see FIGS. 2-3), for each of the first and second flow assemblies 114 and 116, one proportional control valve 118 and six on-off valves 120 are inserted into a single manifold block 20 (for a total of seven valves). Each flow assembly can include two or more (e.g., 2-20) on/off valves. In a preferred embodiment, the proportional control flow valve 118 with a flow range of 0-2 lpms is used to control the flow rate in 0.01 lpms or less increments. The series of six on-off valves 120 with flow rates of 2 lpms, 4 lpms, 8 lpms, 16 lpms, 32 lpms, and 36 lpms can be activated selectively, additively and in parallel with the proportional control valve 118 to achieve various flow rates for the flow assembly between 0-100 lpms. In a preferred embodiment, the first and second flow assemblies 114 and 116 are controlled simultaneously and in parallel, with the first flow assembly 114 being supplied with atmospheric air (a first gas) and the second flow assembly 116 being supplied with pure oxygen (a second gas). The first and second flow assembly output gases are then combined in a combined exhaust channel and to a mixed gas outlet or outlet system port 50 to achieve the desired FiO₂ (fraction of inspired oxygen or the concentration of oxygen that a person inhales) concentrations ranging from 21% (i.e., normal atmospheric air's 21% oxygen concentration, where only the valves of the manifold block or first flow assembly 114 supplied by atmospheric air are open) up to 100% (where only the valves of the manifold block or second flow assembly 116 supplied by pure oxygen are open).

With reference to FIGS. 2-3, in an exemplary use of the mixing assembly 112 combining air and oxygen is described. Inlet air pressure (a first gas), for example between the pressures of 35-90 psig, enters the first flow assembly 114 through an inlet 35. A pressure regulator component 36 reduces the air pressure to the desired input pressure (e.g., 30 psig) to maintain a constant pressure for the input pressure to the on/off valves 120 and proportional valve 118. The outlet 37 of the pressure regulator component 36 is in pneumatic communication with the inlet ports 38 of the on/off valves 120 and the inlet port 39 of the proportional valve 118. In FIG. 2, the flow path of the first gas through the on/off valves 120 is represented by the arrow type labeled A1 and may be referred to herein as the first gas on/off flow path. The 2 lpms, 4 lpms, 8 lpms, 16 lpms, 32 lpms, and 36 lpms on/off valves 120 are opened as needed to achieve the desired on/off flow rate through on/off combined outlet 41. The flow path of the first gas through the proportional valve 118 is represented by the arrow type labeled A2 and may be referred to herein as the first gas proportional flow path.

Inlet oxygen pressure (a second gas), for example between the pressures of 35-90 psig, enters the second flow assembly 116 through an inlet 35. A pressure regulator component 36 reduces the oxygen pressure to the desired input pressure (e.g., 30 psig) to maintain a constant pressure for the input pressure to the on/off valves 120 and proportional valve 118. The outlet 37 of the pressure regulator component 36 is in pneumatic communication with the inlet ports 38 of the on/off valves 120 and the inlet port 39 of the proportional valve 118. In FIG. 2, the flow path of the second gas through the on/off valves 120 is represented by the arrow type labeled A1 and may be referred to herein as the second gas on/off flow path. The 2 lpms, 4 lpms, 8 lpms, 16 lpms, 32 lpms, and 36 lpms on/off valves 120 are opened as needed to achieve the desired on/off flow rate through on/off combined outlet 41. The flow path of the second gas through the proportional valve 118 is represented by the arrow type labeled A2 and may be referred to herein as the first gas proportional flow path.

In each of the first and second flow assemblies, the outlet ports 40 of the on/off valves 120 (through combined outlet 41) and the outlet port 42 of the proportional valve 118 are in pneumatic communication with each other (see conduits 44, which may be referred to as either the first gas output outlet or second gas output outlet). The combined first gas flow (the output of the first flow assembly 114) and the combined second gas flow (the output of the second flow assembly 116) are represented by the arrow type labeled A3 in FIG. 2 and may be referred to as the first gas output path and the second gas output path, respectively. The combined first gas flow and the combined second gas flow join together to create a total hybrid oxygen flow for the system, which exits through mixed gas outlet or outlet system port 50. The combined first and second gas flows (the exhaust gas that is outputted at a final concentration through the exhaust channel, mixed gas outlet or outlet system port 50) is represented by the arrow type labeled A4 in FIG. 2 and may be referred to herein as the mixed gas outlet path.

As discussed above, the first and second gases can be any gases that are desired to be mixed and outputted at a predetermined or desired concentration. However, in the exemplary embodiment shown herein, the combined gases are oxygen and air that are used in and exhausted by the ventilator assembly 110. In a situation where a patient is placed on a ventilator that uses the ventilator assembly 110 of the present invention, the attending doctor provides the desired or predetermined exhaust gas flow rate and the desired or predetermined FiO₂ (or “oxygen concentration”) of the exhaust gas that is delivered to the patient. In order to determine the proper flow rate of oxygen (referred to herein as the “oxygen flow assembly output flow rate”) to be delivered by the second flow assembly 116, the mixing system 112 utilizes the following equation: Oxygen flow assembly output flow rate=predetermined exhaust gas flow rate×(predetermined FiO₂−21%)/79%. Using this equation, the controller of the mixing system (and the related software) opens the proper on/off valves and the proportional valve in the second flow assembly 116, to achieve the desired “oxygen flow assembly output flow rate.” After the oxygen flow assembly output flow rate has been determined, the air flow assembly output flow rate can then be determined by the following equation: Air flow assembly output flow rate=predetermined exhaust flow rate−oxygen flow assembly output flow rate. Using this equation, the controller of the mixing system (and the related software) performs the necessary calculations to determine the proper on/off valves and/or the proportional valve to be opened in the first flow assembly 114, to achieve the desired “air flow assembly output flow rate.” The oxygen and air from the two flow assemblies are then combined and exhausted where they can be delivered to the patient. In other words, adding the oxygen flow assembly output flow rate to the air flow assembly output flow rate results in the predetermined exhaust flow rate (oxygen flow assembly output flow rate+air flow assembly output flow rate results=predetermined exhaust flow rate).

Below are examples showing how to determine oxygen flow assembly and air flow assembly flow rates for a desired gas oxygen concentration (predetermined FiO₂).

Example 1 where the goal is to deliver a predetermined exhaust gas flow rate of 100 lpm with a predetermined FiO₂ of 60.2%. By use of the equation discussed above, we determine oxygen flow rate or oxygen flow assembly output rate to be: (100 lpm)×(60.2%−21%)/79%=50 lpm. The controller calculates or determines which on/off valves and/or the proportional valve to open to achieve 50 lpm. Therefore, to achieve 50 lpm of oxygen flow through the second flow assembly, the 2 lpm, 16 lpm, and the 32 lpm on/off valves are activated to create a total oxygen flow of 50 lpm. In this example, the proportional valve was not needed to be opened.

The air flow rate required is 100 lpm (the predetermined exhaust gas flow rate) minus the 50 lpm oxygen flow assembly output flow rate. Therefore, the first flow assembly 114 must deliver an air flow assembly output flow rate of 50 lpm to meet the predetermined exhaust gas flow rate of 100 lpm. To achieve 50 lpm of air flow, the 2 lpm, 16 lpm, and the 32 lpm on/off valves are activated to create a total air flow of 50 lpm. It will be appreciated that a FiO₂ of 60.2% is the only FiO₂ occurrence when the two gases (oxygen and air) have the same output flow.

Example 2 where the goal is to deliver 100 lpm at an FiO₂ of 30% out of the mixing system outlet port or combined exhaust channel and to the ventilator. Solving the following equation yields the oxygen flow: Oxygen flow assembly output flow rate=(100 lpm)×(30%−21%)/79%=11.4 lpm. The controller determines which on/off valves and/or the proportional valve to open to achieve 11.4 lpm of oxygen flow. In this example, in the second flow assembly 114, the on/off valves in conjunction with the proportional valve have to be used. The flow is created by the use of the on/off valve assemblies creating the integer flow. This is accomplished by the use of the 8 lpm and the 2 lpm on/off valves. Now that the 8 lpm and the 2 lpm on/off valves are delivering a total of 10 lpm the remaining 11.4−10=1.4 lpm is delivered by the proportional valve. The air flow rate required is 100 lpm (the predetermined exhaust gas flow rate) minus the 11.4 lpm oxygen flow assembly output flow rate, which equals 88.6 lpm. This can be provided by opening the 36 lpm, 32 lpm, 16 lpm and 4 lpm on/off valves (for 88 lpm of flow) together with the proportional valve delivering 0.6 lpm. The 11.4 lpm oxygen flow assembly output flow rate together with the 88.6 lpm air flow assembly output flow rate deliver 100 lpm of combined gas at an FiO₂ of 30%.

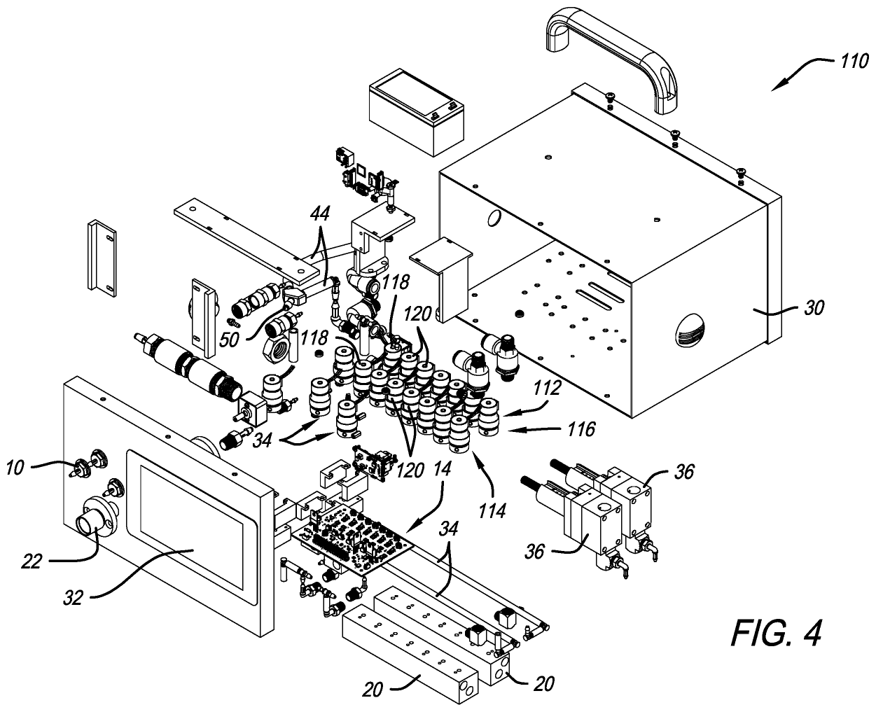

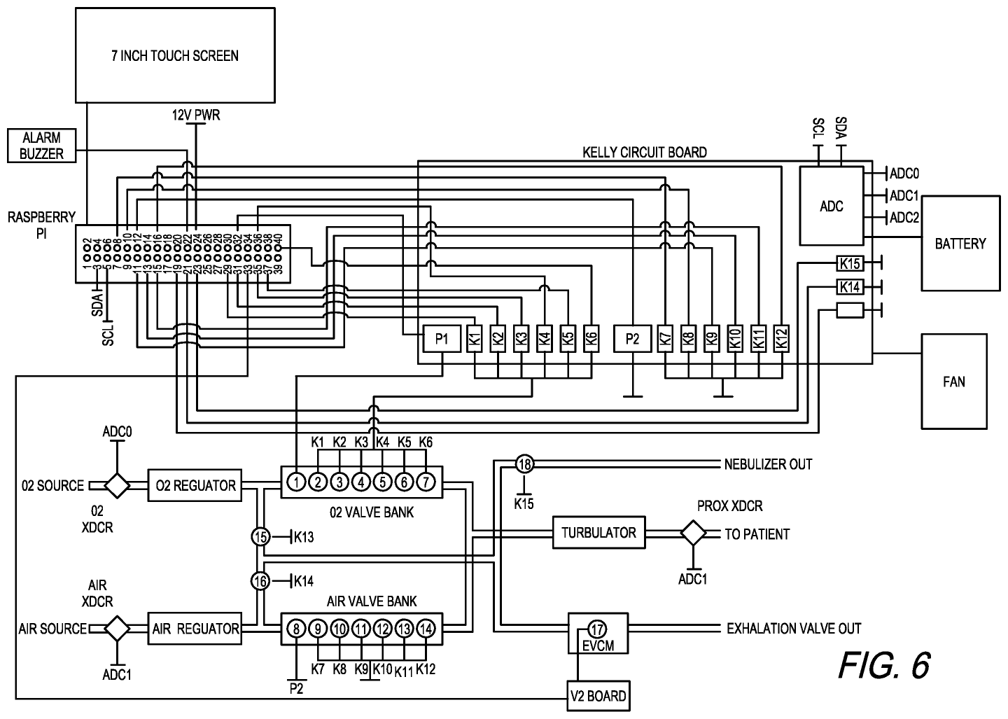

FIG. 4 shows an exploded view of the ventilator assembly 110. It will be appreciated by those of ordinary skill in the art that most of the components in the ventilator assembly 110, other than the mixing system 112, are known and are therefore not described in detail herein. For example, the mixing system 112 provides an output or mixed gas that is through an outlet 22 in the ventilator assembly 110. However, as shown in FIG. 4, various other components in the ventilator assembly are utilized in prior art ventilators. For example components 34 are used as part of the control of the exhalation valve 10. The ventilator assembly 110 also generally includes a housing 30, screen 32, an outlet 22 (through which the mixed gas is exhausted), exhalation valve 10 and inputs or inlets for connecting the oxygen and air for mixing. It will be appreciated that the ventilator assembly 10 (and mixing system 112) includes electronics, circuitry and the like therefor. Much of this is shown in PCB or motherboard 14 in FIG. 4 (see also FIG. 6). The controller may be a part of the PCB. The mixing system 112 and ventilator assembly 110 also include various conduits, pipes or the like through which the first and second gases flow. The types of conduits, pipes, etc. are not a limitation on the present invention and are generally labeled 34 in the drawings.

The overall ventilator system within which the ventilator assembly 110 is utilized may include a mask, oxygen monitor, artificial lung, appropriate tubes and valves and other components typically utilized in a ventilator system for a patient.

It should be remembered that a flow sensor is preferably not utilized in this system (However, this is not a limitation and a flow sensor can be used). Therefore, the proportional valve is considered to be in an open loop state. However, since the valve only delivers a maximum flow of 2 lpm, the proportional valve flow is minimal to the total flow. Thus, it is satisfactory enough in accuracy to not cause substantial errors to flow or FiO₂ accuracy.

It has been determined that proportional valves in an open loop condition can repeat flow versus electrical current within 20% of the maximum flow of the valve. This yields a tolerance of +/−0.4 lpm error to flow throughout the entire flow regime of the valve, which is 0-2 lpm. Therefore, if electrical current is varied to the proportional valve it can deliver a fairly consistent repeatable flow throughout the flow range without the use of a flow sensor in a closed looped application. Based on a 20% proportional flow error, the desired flow of 1.4 lpm can vary from 1.0-1.8 lpm. This will not create a substantial FiO₂ error and is considered acceptable to industry standards of most applications.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶6, other aspects may likewise be embodied as a means-plus-function claim, or in other forms, such as being embodied in a computer-readable medium. (Any claims intended to be treated under 35 U.S.C. § 112, ¶6 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A gas mixing system comprising:
a first flow assembly that includes an inlet for receiving a first gas, a regulator, a plurality of on/off valves connected in series and a proportional valve, wherein each on/off valve includes a different on/off flow rate value and is selectively openable to allow the first gas to flow therethrough, wherein a first gas on/off flow path is defined from the inlet, through the regulator, through at least one of the on/off valves and to a first gas on/off outlet, wherein a first gas proportional flow path is defined from the inlet, through the regulator, through the proportional valve and to a first gas proportional outlet, wherein the first gas on/off flow path and first gas proportional flow path combine to create a first gas output path,
a second flow assembly that includes an inlet for receiving a second gas, a regulator, a plurality of on/off valves connected in series and a proportional valve, wherein each on/off valve includes a different on/off flow rate value and is selectively openable to allow the second gas to flow therethrough, wherein a second gas on/off flow path is defined from the inlet, through the regulator, through at least one of the on/off valves and to a second gas on/off outlet, wherein a second gas proportional flow path is defined from the inlet, through the regulator, through the proportional valve and to a second gas proportional outlet, wherein the second gas on/off flow path and second gas proportional flow path combine to create a second gas output path, and
an exhaust gas outlet, wherein the first gas output path and second gas output path combine to create an exhaust gas outlet path that extends through the exhaust gas outlet.

2. The gas mixing system of claim 1 wherein the proportional valve in the first flow assembly includes a range of proportional flow rate values, wherein the plurality of on/off valves in the first flow assembly are each configured to output an on/off flow rate value that is an integer, and wherein the proportional valve in the first flow assembly is configured to output a proportional flow rate value that is a fraction.

3. The gas mixing system of claim 1 further comprising a controller, wherein the first gas is air and the second gas is oxygen.

4. A ventilator assembly that includes the gas mixing system of claim 3.
